(19) United States
(12) Patent Application Publication
(10) Pub. No.: US 2025/0188028 A1
(43) Pub. Date: Jun. 12, 2025

(54) Synthetic Method for Producing Ionizable Amino Lipids

(71) Applicant: NanoVation Therapeutics Inc., Vancouver (CA)

(72) Inventors: Fariba Saadati, Vancouver (CA); N. D. Prasad Atmuri, Vancouver (CA); Marco A. Ciufolini, Vancouver (CA)

(73) Assignee: NanoVation Therapeutics Inc., Vancouver, BC (CA)

(21) Appl. No.: 18/845,589

(22) PCT Filed: Mar. 6, 2023
(86) PCT No.: PCT/CA2023/050287

§ 371 Date: Sep. 10, 2024

Related U.S. Application Data
(60) Provisional application No. 63/269,297, filed on Mar. 14, 2022.

(51) Int. Cl. C07D 207/16; C07C 67/08; C07C 227/08

(52) U.S. Cl. CPC C07D 207/16; C07C 67/08; C07C 227/08

(57) ABSTRACT

Provided herein is a method for a method for producing an ionizable amino lipid, or a pharmaceutically acceptable salt thereof, the method comprising a nucleophilic displacement of a leaving group L in a compound of Formula II, wherein G¹ is (CH₂)ₙ, wherein n is 2 to 10, and wherein R¹ and R² are independently substituted, unsubstituted, branched or unbranched C₁-C₂₀ alkyl having 0 to 3 double bonds, with an amino alcohol, resulting in an N-double alkylation of the amino alcohol by the compound of Formula II to form the ionizable amino lipid.

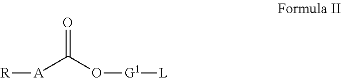

Further provided are intermediates of Formula II and methods for preparing the intermediates by an esterification step.

### CROSS-REFERENCE TO RELATED APPLICATIONS

This application claims priority from U.S. Ser. No. 63/269,297 filed on Mar. 14, 2022, which is incorporated herein by reference in its entirety.

### TECHNICAL FIELD

Provided herein is a method for the chemical synthesis of an ionizable lipid.

### BACKGROUND

Nucleic acid-based therapeutics have enormous potential in medicine. To realize this potential, however, the nucleic acid must be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles have been developed that protect nucleic acid from such degradation and facilitate delivery across cellular membranes to gain access to the intracellular compartment, where the relevant translation machinery resides.

A key component of lipid nanoparticles is an ionizable lipid. The ionizable lipid is typically positively charged at low pH, which facilitates association with the negatively charged nucleic acid. However, the ionizable lipid is neutral at physiological pH, making it more biocompatible in biological systems. Further, it has been suggested that after the lipid nanoparticles are taken up by a cell by endocytosis, the ionizability of these lipids at low pH enables endosomal escape. This in turn enables the nucleic acid to be released into the intracellular compartment.

An earlier example of a lipid nanoparticle product approved for clinical use and reliant on ionizable lipid is Onpattro®, developed by Alnylam. Onpattro® is a lipid nanoparticle-based short interfering RNA (siRNA) drug for the treatment of polyneuropathies induced by hereditary transthyretin amyloidosis. Onpattro® is reliant on an ionizable lipid referred to as “DLin-MC3-DMA” or more commonly “MC3”, 1 (FIG. 1), by investigators. Furthermore, MC3 represents an evolution of a structurally related ionizable lipid, referred to by investigators as “KC2”, 2 (FIG. 1). MC3 is a state-of-the art ionizable lipid for the delivery of siRNA, having been found to require about 3 times less siRNA than KC2, but KC2 is superior in other applications, and it remains a valuable research tool.

While the foregoing ionizable lipids are especially efficacious for the delivery of siRNA-containing LNPs to hepatic cells, they are much less effective for the hepatic delivery of mRNA-containing LNPs. To illustrate, mRNA vaccines, including the COVID-19 Pfizer/BioNTech and Moderna vaccines, rely on lipid nanoparticles to deliver mRNA to the cytoplasm of liver cells. After entry into the host cell, the mRNA is transcribed to produce antigenic proteins. In the case of the COVID-19 vaccines, the mRNA encodes the highly immunogenic Sars-Cov-2 spike protein. Such vaccines, however, incorporate no MC3 or KC2, but other types of ionizable lipids. In particular, the Pfizer/BioNTech vaccine comprises an ionizable lipid referred to as “ALC-0315”, 3 (Scheme 1).

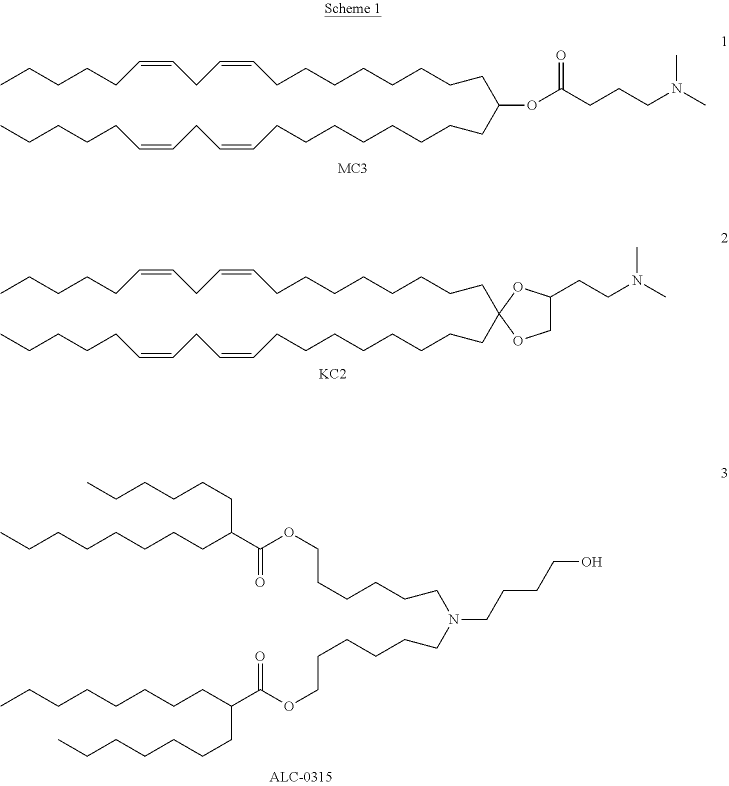

The synthesis of 3 has been described in U.S. Pat. No. 10,166,298. However, the disclosed synthesis is low-yielding and it generates large quantities of byproducts, thereby making the synthesis wasteful. Moreover, the synthesis employs problematic reagents, such as pyridinium chlorochromate (PCC). The PCC compound is a chemical oxidant based on hexavalent chromium [“Cr(VI)”], which is a known carcinogen.

There is a pressing need in the art for a better synthesis of lipids including, but not limited to, ALC-0315, that affords higher yields, is less wasteful, and/or avoids the use of problematic Cr(VI)-based reagents. Such a need remains unmet in the industry. The present disclosure addresses the shortcomings in the art and/or provides an alternative synthetic process that circumvents one or more of the above difficulties.

### SUMMARY

The present disclosure provides methods for the preparation of amino lipids. Examples include ALC-0315 [systematic name: ((4-hydroxybutyl)azanediyl)bis(hexane-6,1-diyl)bis(2-hexyldecanoate)] and other lipids, including but not limited to those that are structurally similar to ALC-0315.

According to one aspect of the disclosure, there is provided a method for producing an ionizable amino lipid, or a pharmaceutically acceptable salt thereof, the method comprising a nucleophilic displacement of a leaving group L in a compound of Formula II,

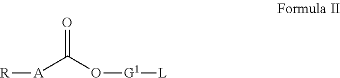

wherein R is a linear or branched C₈ to C₃₀ alkyl group, optionally comprising heteroatoms selected from N, O and/or S, optionally comprising 1-3 C═C double bonds of Z or E geometry, optionally comprising cyclic structures, and/or optionally comprising amide and/or ester linkages, A is absent or present, and, when present, is a moiety derived from an aminoacid, optionally selected from proline, sarcosine, alanine, beta-alanine and optionally representable as Formula III:

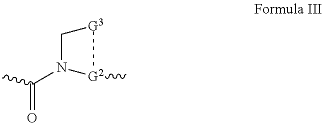

wherein G² and G³ are bonded to each other or not bonded, and
if G² and G³ are bonded to each other, then G² is CH, G² is bonded to the carbonyl (C═O) group in Formula II, G³ is (CH₂)ₙ wherein n is 1 to 4, and the C═O group in Formula III is bonded to the R group in Formula II, and
if G² and G³ are not bonded, then G² is (CH₂)ₚ wherein p is 1 to 3, G² is bonded to the carbonyl (C═O) group in Formula II, G³ is (CH₂)_{q}—H wherein q is 0 to 3, and the C═O group in Formula III is bonded to the R group in Formula II, G¹ is (CH₂)ₙ, wherein n is 2 to 10,
wherein the leaving group L is a halogen atom selected from chlorine, bromine, or iodine or a sulfonate selected from a tosylate or a mesylate, wherein the nucleophilic displacement comprises reacting a compound of Formula II with an amino alcohol in a single alkylation reaction, resulting in an N-double alkylation of the amino alcohol by the compound of Formula II to form the ionizable amino lipid; or wherein the nucleophilic displacement comprises reacting two different compounds of Formula II in sequential displacement reactions, a first reaction comprising reacting the amino alcohol with a first compound of Formula II to produce a single N-alkylated compound and alkylating the single N-alkylated compound so produced in a second reaction with a second compound of Formula II to produce the ionizable lipid.

According to one embodiment, the n of (CH₂)ₙ in G¹ is 2 to 8.

According to another embodiment, the R of Formula II is a branched chain that comprises two hydrophobic carbon chains, R¹ and R², each of the chains being independently linear, unsubstituted C₂ to C₁₂ alkyl with 0 double bonds and wherein a carbon branch point is at an alpha, beta or gamma position relative to the carbonyl group of Formula II.

According to a further embodiment, the nucleophilic displacement comprises reacting a compound of Formula II with an amino alcohol in the single alkylation reaction and wherein Formula II has the structure of Formula I below:

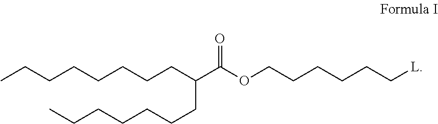

According to a further embodiment, the nucleophilic displacement comprises reacting the first and second compounds of Formula II in the sequential displacement reactions and wherein the first or second compound of Formula II has a structure of Formula IV below:

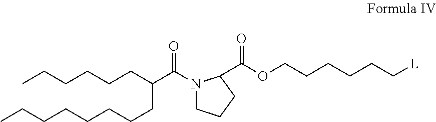

and wherein the first or second compound of Formula II has a structure as in Formula I

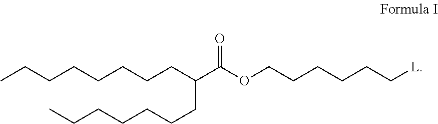

According to a further embodiment, the amino alcohol is 4-amino-1-butanol.

In yet a further embodiment, the nucleophilic displacement is carried out in a polar solvent with a base and optionally with a source of iodide ion.

According to a further embodiment, the base is a metal carbonate.

According to another embodiment, the optional source of iodide ion is lithium iodide, sodium iodide, potassium iodide, or a tetraalkylammonium iodide.

According to another embodiment, the method further comprises producing the compound of Formula II via an esterification reaction between a carboxylic acid having the structure of Formula V, wherein R¹ and R² are independently substituted, unsubstituted, branched or unbranched C₁-C₂₀ alkyl having 0 to 3 double bonds, and an alcohol with a terminal leaving group L as defined by the structure of Formula VI, wherein G¹ is (CH₂)ₙ, and wherein n is 2 to 10 and L is the leaving group

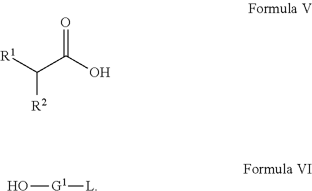

In yet another embodiment, the method further comprises producing the compound of Formula II via an esterification reaction between a carboxylic acid having a structure of Formula V, wherein R¹ and R² are independently substituted, unsubstituted, branched or unbranched C₁-C₂₀ alkyl having 0 to 3 double bonds, and a hydroxyl group of a diol having the structure of Formula VII, wherein G¹ is (CH₂)ₙ, and wherein n is 2 to 10; and wherein

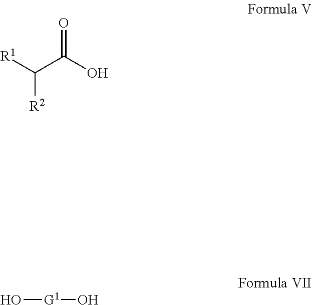

the esterification reaction produces a compound having a structure of Formula VIII:

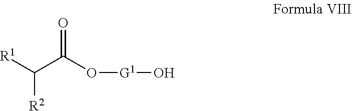

and converting the OH of the compound of Formula VIII into the leaving group L to produce a compound having a structure of Formula II.

According to a further embodiment, the esterification reaction is a Fischer esterification that is carried out in an inert solvent capable of forming an azeotrope with water.

According to another embodiment, the inert solvent is toluene, cyclohexane, 1,2-dichloroethane.

According to a further embodiment, the water produced during the esterification is removed such that the carboxylic acid serves as an acid catalyst for the esterification reaction.

According to a further embodiment, an acid catalyst is used in the esterification, the acid catalyst selected from sulfuric acid, phosphoric acid, para-toluenesulfonic acid, sodium hydrogen sulfate, or a sulfonated polystyrene resin.

In a further embodiment, the nucleophilic displacement comprises the single alkylation and wherein the ionizable lipid produced is ALC-0315.

According to another aspect, there is provided an intermediate for preparing an amino ionizable lipid, the intermediate having a structure of Formula I:

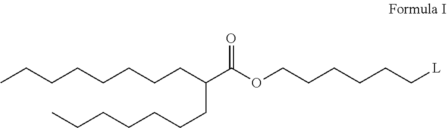

wherein L is a halogen atom selected from chlorine, bromine, or iodine or a sulfonate selected from a tosylate or a mesylate.

According to another aspect, there is provided an intermediate for preparing an amino ionizable lipid, the intermediate having a structure of Formula IX,

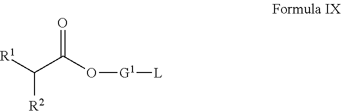

wherein L is a halogen atom selected from chlorine, bromine, or iodine or a sulfonate selected from a tosylate or a mesylate.

According to another aspect, there is provided an intermediate for preparing an amino ionizable lipid, the intermediate having a structure of Formula X below:

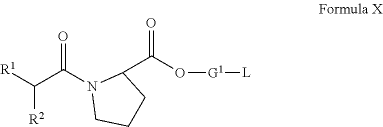

wherein L is a halogen atom selected from chlorine, bromine, or iodine or a sulfonate selected from a tosylate or a mesylate.

According to another embodiment, the L of either of the foregoing intermediates is bromine, chlorine, a tosylate or a mesylate.

According to another aspect, there is provided a method for preparing the intermediate of any one of the aspects or embodiments described above, comprising:
(a) an esterification between a hydroxyl group and a carboxylate group of the compounds of Formula V and Formula VI to produce the intermediate

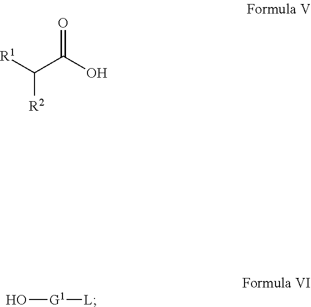

or
(b) an esterification between a hydroxyl group and a carboxylate group of the compounds of Formula V and Formula VII to produce a compound of Formula VIII and further reacting the compound of Formula VIII to convert the terminal hydroxyl group into a leaving group to produce the intermediate

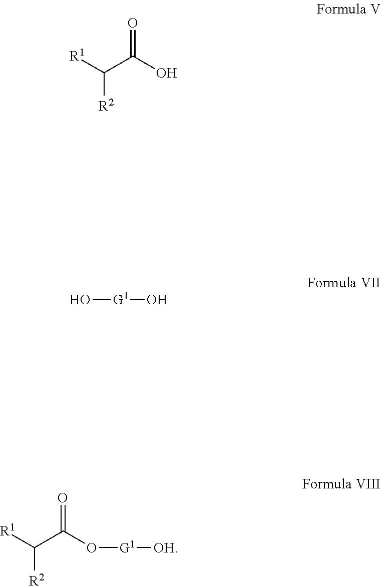

According to a further embodiment, the esterification reaction is a Fischer esterification that is carried out in an inert solvent capable of forming an azeotrope with water.

According to a further embodiment, the inert solvent is toluene, cyclohexane, 1,2-dichloroethane.

According to another embodiment, the water produced during the esterification is removed such that the carboxylic acid serves as an acid catalyst for the esterification reaction.

In a further embodiment, an acid catalyst is used in the esterification, the acid catalyst selected from sulfuric acid, para-toluenesulfonic acid, sodium hydrogen sulfate, or a sulfonated polystyrene resin.

In some non-limiting embodiments, the foregoing methods comprise the nucleophilic displacement of a leaving group L in a compound of Formula I with 4-amino-1-butanol, resulting in formation of an amino lipid, such as ALC-0315 or structurally related ionizable lipids.

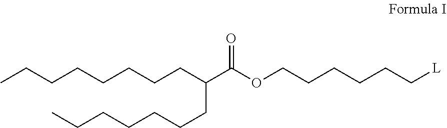

Group L can be a halogen, such as bromide, or a sulfonate, such as mesylate or tosylate. Furthermore, the nucleophilic displacement is carried out in an appropriate solvent, at an appropriate temperature, and in the presence of a base such as a metal carbonate.

Provided herein are also methods for the preparation of compounds of Formula I wherein L=Br or L=O—SO₂-4-tolyl (tosylate) or L=O—SO₂—CH₃ (mesylate).

Compounds of Formula I wherein L=halogen (bromide, chloride) can be prepared by Fischer esterification of a 6-halohexanol, for example, 6-bromohexanol, with 2-hexyldecanoic acid, leading to 6-bromohexyl-2-hexyldecanoate. This reaction can be carried out in an inert solvent having an appropriate boiling point and capable of forming an azeotrope with water, such as toluene, cyclohexane, 1,2-dichloroethane, and the like, whereupon the use of an apparatus for continuous removal of the water produced in the course of the esterification process, for example, a Dean-Stark trap, allows 2-hexyldecanoic acid itself to serve as the acid catalyst for the esterification reaction, thereby suppressing the need for an additional acid catalyst. However, an acid catalyst such as sulfuric acid, para-toluenesulfonic acid, sodium hydrogen sulfate, or sulfonated polystyrene resins, may be optionally employed as catalysts in the esterification step.

Compounds of Formula I wherein L=O—SO₂-4-tolyl (tosylate) or L=O—SO₂—CH₃ (mesylate) can be prepared by Fischer esterification of 1,6-hexanediol with 2-hexyldecanoic acid, leading to 6-hydroxyhexyl-2-hexyldecanoate. This reaction can be carried out in an inert solvent of appropriate boiling point and capable of forming an azeotrope with water, such as toluene, cyclohexane, 1,2-dichloroethane, and the like, whereupon the use of an apparatus for continuous removal of the water produced in the course of the esterification process, for example, a Dean-Stark trap, allows 2-hexyldecanoic acid itself to serve as the acid catalyst for the esterification reaction, thereby suppressing the need for an additional acid catalyst. However, an acid catalyst such as sulfuric acid, para-toluenesulfonic acid, sodium hydrogen sulfate, or sulfonated polystyrene resins, may be optionally employed as catalysts in the esterification step. The OH group in the 6-hydroxyhexyl-2-hexyldecanoate thus produced is subsequently converted into a sulfonate ester, such as a tosylate or a mesylate, by reaction with tosyl chloride or mesyl chloride in an appropriate solvent, such as CH₂Cl₂, and in the presence of a base such as triethylamine and optionally of a catalyst such as 4-dimethylaminopyridine (DMAP).

Advantages of the synthesis schemes of the present disclosure include in some embodiments higher overall yields (60-70% instead of 15-20% achieved with a known process described herein) and/or method steps that: (a) avoid use of coupling agents; (b) eschew hazardous reagents such as PCC, which contains carcinogenic Cr(VI); (c) bypass the need for redox operations, (d) are easier to implement, (e) reduce the amount of waste generated, and/or (f) proceed more cleanly, greatly facilitating the purification of the final product, thus realizing significant economies in terms of solvents, chromatographic supports, and plant and operator time.

The above summary is intended to illustrate non-limiting embodiments of the disclosure but is in no way intended to limit the scope of the invention.

### BRIEF DESCRIPTION OF THE DRAWINGS

FIG. 1 depicts the synthesis scheme of ALC-0315 as described by in the prior art (U.S. Pat. No. 10,166,298).

FIG. 2 depicts a method for the synthesis of ALC-0315 comprising a nucleophilic displacement of bromide, as described in the present disclosure.

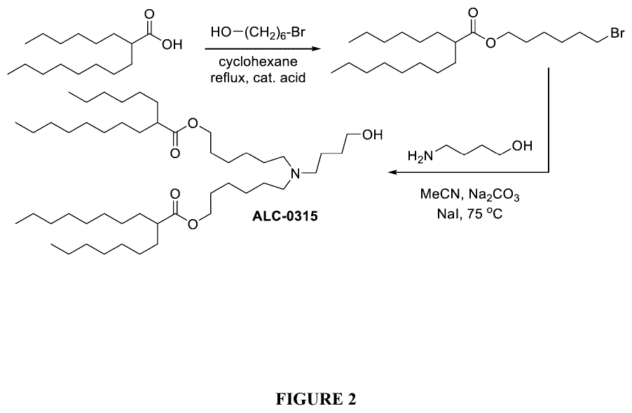

FIG. 3 depicts a method for the synthesis of ALC-0315 comprising a nucleophilic displacement of mesylate, as described in the present disclosure.

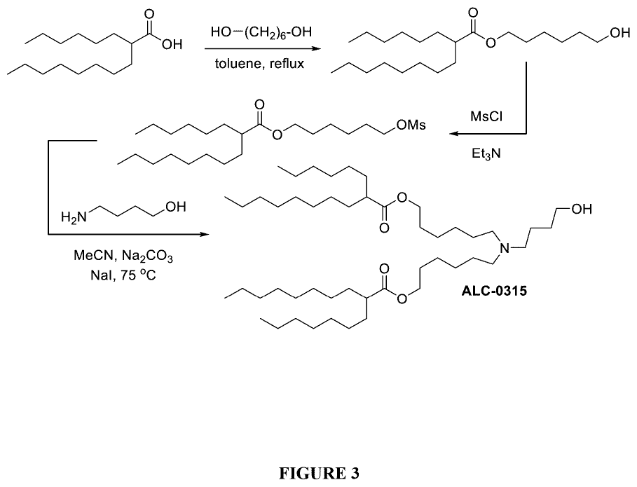

FIG. 4 depicts a method for the synthesis of an unsymmetrical ionizable lipid comprising two sequential nucleophilic displacements, as described in the present disclosure.

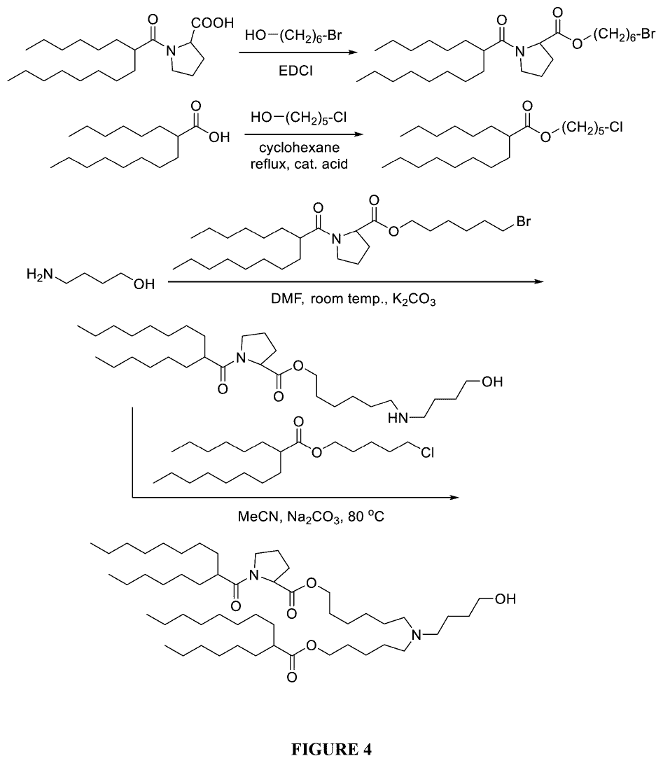

### DETAILED DESCRIPTION

The detailed description and examples below are intended to illustrate non-limiting embodiments of the disclosure but are in no way intended to limit the scope of the invention.

Provided herein is a method for the chemical synthesis of ionizable amino lipids, including a lipid known as ALC-0315, which is a component of the Pfizer-BioNTech COVID-19 vaccine. As described above, previous methods of ALC-0315 synthesis are problematic and have low yields. The present disclosure provides a solution to such difficulties and outlines an improved chemical route to produce the ionizable lipid as well as other related compounds.

As those of ordinary skill in the art will appreciate, the reactions employed herein may be carried out in any appropriate solvent, or mixtures of solvents, and at appropriate temperatures.

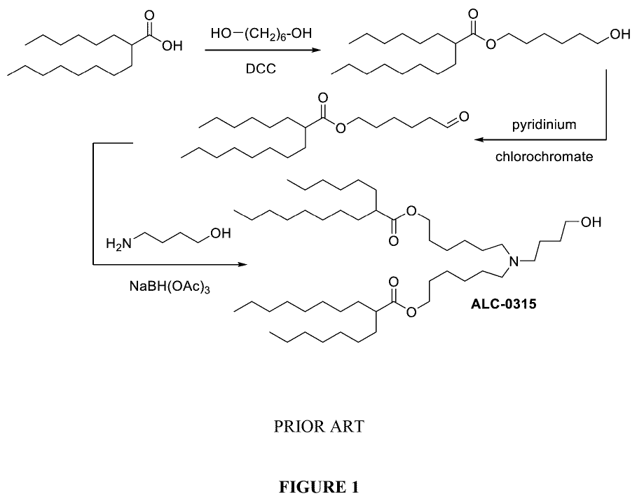

The known synthesis of ALC-0315 (FIG. 1) comprises three steps, among which step 1 is efficient and has an acceptable yield. However, the inventors have recognized that this step would become even more efficient if the use of a coupling agent could be avoided. This would eliminate or reduce the use of extra reagents and simplify the isolation of the product.

Furthermore, steps 2 and 3 of the synthesis of FIG. 1 are problematic. Step 2 requires the use of PCC, which is an oxidant based on the carcinogenic compound, Cr(VI). The hazards associated with the use of PCC in a pharmaceutical plant and the cost of disposal of the reagent and of its waste products limit its use in a pharmaceutical production facility. Furthermore, aldehyde 7 emerging from the reaction is accompanied by numerous contaminants. The inventors' own observations suggest that the contaminants accompanying 7 arise from 7 itself, through self-condensation reactions promoted by PCC (see discussion above). Aldehyde 7 thus requires purification by chromatography. However, the preparation of 7 constitutes a relatively early step of the synthesis. This translates into a need to purify large quantities of 7 by chromatography. Such a requirement introduces significant technical difficulties into the manufacturing process of 7 and greatly increases the cost of production. The inventors have recognized that a better process avoids chromatography at such an early stage.

Step 3 of FIG. 1, considered by the inventors the most troublesome step in the sequence, is stated to produce ALC-0315 at about 20% yield. This step produced ALC-0315 contaminated with a multitude of side products and imposes the need for extensive chromatographic purification of the desired 3, with an attendant requirement for considerable operator time and large quantities of solvents and chromatographic supports. This results in the generation of significant waste that ultimately should be disposed of. As a consequence, this step has economics that are unsustainable.

The inventors recognize that a more desirable pharmaceutical process avoids or reduces the use of reduction or oxidation (“redox”) reactions. In this regard, the present disclosure provides, in some non-limiting embodiments, a method comprising a two-or three-step synthesis that avoids (i) the use of a coupling agent in a first step of the method; (ii) the oxidation of an alcohol to an aldehyde; and (iii) the reductive amination reaction. Furthermore, the method may only necessitate chromatographic operations for the purification of the final product.

The synthesis method in some embodiments comprises an initial Fischer esterification of 2-hexyldecanoic acid, 4, with 6-bromohexanol, 5, leading to 6-bromohexyl-2-ethyldecanoate, 7. While Fischer esterification reactions are often carried out in the presence of a strong Brønsted acid catalyst such as HCl, H₂SO₄, H₃PO₄, TsOH, sodium hydrogen sulfate, sulfonated polystyrene (Dowex® IR resin), and the like, the inventors have determined that such catalysts are unnecessary in embodiments of the present method. Thus, refluxing a solution of 2-hexyldecanoic acid and 6-bromohexanol in a solvent that forms an azeotrope with water, for example, toluene, cyclohexane, or 1,2-dichloroethane, with continuous removal of water, for example by the use of a Dean-Stark trap, results in quantitative formation of 6-bromohexyl 2-hexyldecanoate, in that the 2-hexyldecanoic acid itself functions as the acid catalyst for the reaction. However, the esterification reaction can be optionally carried out in the presence of a small quantity of a non-volatile acid, such as H₂SO₄, H₃PO₄, TsOH, sodium hydrogen sulfate, or a sulfonated polystyrene resin. In either case, the yield of 6-bromohexyl-2-ethyldecanoate is 95-97% (Scheme 2).

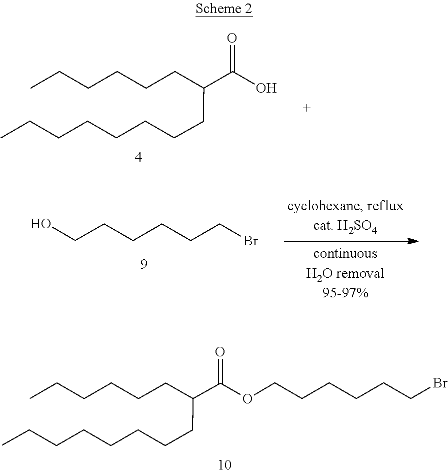 [SMILES: CCCCCCCCC(CCCCCC)C(=O)O ; OCCCCCCBr ; CCCCCCCCC(CCCCCC)C(=O)OCCCCCCBr]

In alternative embodiments, the synthesis may start with the Fischer esterification of 2-hexyldecanoic acid with 1,6-hexanediol, leading to 6-hydroxyhexyl-2-ethyldecanoate. While Fischer esterification reactions are often carried out in the presence of a strong Brønsted acid catalyst such as HCl, H₂SO₄, H₃PO₄, TsOH, sodium hydrogen sulfate, sulfonated polystyrene (Dowex® IR resin), and the like, the inventors have determined that such catalysts are unnecessary in certain embodiments. Thus, refluxing a solution of 2-hexyldecanoic acid and 1,6-hexanedol in a solvent that forms an azeotrope with water, for example, toluene, cyclohexane, or 1,2-dichloroethane, with continuous removal of water, for example by the use of a Dean-Stark trap, results in quantitative formation of 6-bromohexyl 2-hexyldecanoate, in that the 2-hexyldecanoic acid itself functions as the acid catalyst for the reaction. However, the esterification reaction can be optionally carried out in the presence of a small quantity of a non-volatile acid, such as H₂SO₄, H₃PO₄, TsOH, sodium hydrogen sulfate, or a sulfonated polystyrene resin. In either case, the yield of 6-hydroxyhexyl-2-ethyldecanoate is 95-97%.

The free OH group in the 6-hydroxyhexyl 2-hexyldecanoate thus obtained is activated toward nucleophilic displacement by conversion into a good leaving group. Such a leaving group may be a sulfonate ester such as a tosylate, a mesylate, a triflate, a 4-nitrobenzenesulfonate, and the like. The sulfonate esters above may be prepared by reaction of the alcohol with an appropriate sulfonyl chloride or sulfonic anhydride. For example, and without intending to be limiting, the reaction of 6-hydroxyhexyl 2-hexyldecanoate with methanesulfonyl chloride (mesyl chloride) produces 6-(mesyloxy)hexyl 2-hexyldecanoate (Scheme 3).

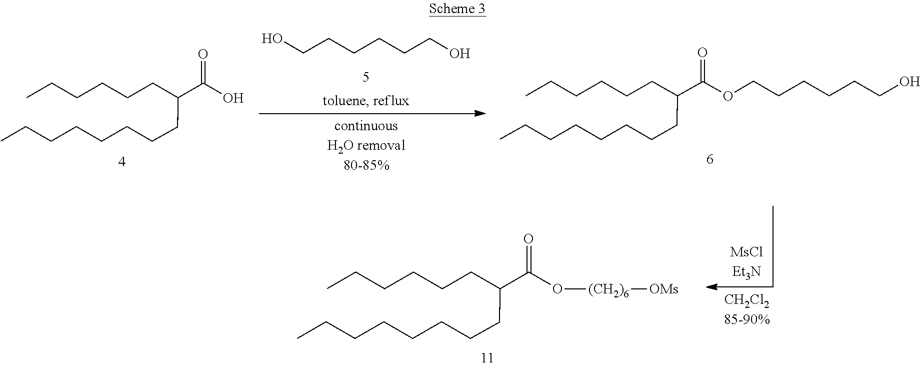

The double N-alkylation of 4-amino-1-butanol with halide 10 or sulfonate 11 is carried out in a polar solvent, at an appropriately elevated temperature typically between 50 and 100° C. and optionally with microwave irradiation, in the presence of a metal carbonate, and optionally in the presence of a source of iodide ion, such as lithium iodide, sodium iodide, potassium iodide, a tetraalkylammonium iodide, and the like. Such a reaction is most advantageously carried out in a solvent in which compounds 8, 10 or 11, and optionally the source of iodide ion are soluble, but in which the product 3 is poorly soluble or insoluble. Accordingly, as the reaction progresses the reaction mixture separates into two layers: one comprising a solution of starting compounds 8, 10 or 11, and optionally the source of iodide ion in the reaction solvent, and one comprised largely of product 3. This greatly facilitates the recovery of the product and its subsequent purification.

Without intending to be limiting, the process is exemplified in Scheme 4 with the reaction of 10 or 11 with 8 in acetonitrile (MeCN) at 75° C. and in the presence of Na₂CO₃, resulting in formation of 3 in 70-80% yield after chromatography.

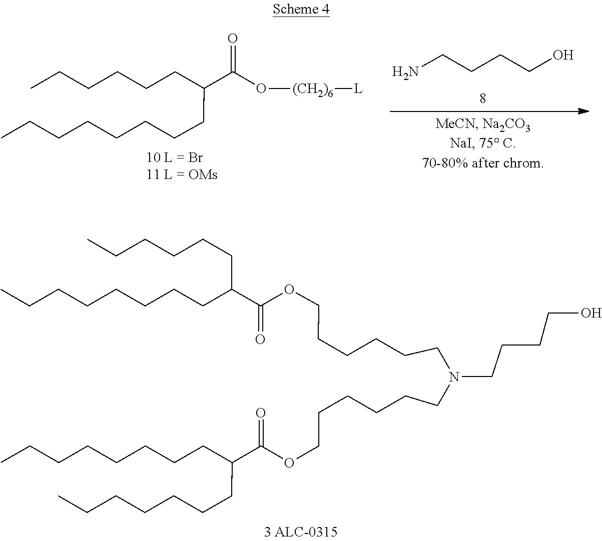

It is apparent from Scheme 4 that the formation of product 3 involves a double N-alkylation of the amine, whereby a pair of identical alkyl groups become bound to the N atom of the starting amine. their will be appreciated by those of skill in the art that the first N-alkylation step, namely, the mono-alkylation of the amine, is faster that the second step, i.e., the dialkylation of the amine. The inventors have discovered a noteworthy solvent-and temperature effect in the relative rates of the two steps. Accordingly, conduct of the reaction in an appropriate solvent at a suitably lower temperature results in selective mono-alkylation of a starting amine such as 8. The product of such a mono-N-alkylation reaction may then be subjected to a second N-alkylation reaction, in another appropriate solvent and at a higher temperature, resulting in formation of a product in which two different alkyl groups have become bound to the N atom of the starting amine.

Without intending to be limiting, this is exemplified in Scheme 5 with the synthesis of ionizable lipid 15, which is also described in co-pending and co-owned U.S. provisional patent application No. 63/410,273 filed on Sep. 27, 2022. Thus, the reaction of amine 8 with alkyl bromide 12, in N,N-dimethylformamide (DMF), at room temperature and in the presence of K₂CO₃ produces largely mono-alkylated product 13. Further reaction of 13 with alkyl chloride 14 in MeCN at 80° C. and in the presence of Na₂CO₃, optionally with microwave irradiation, results in formation of lipid 15.

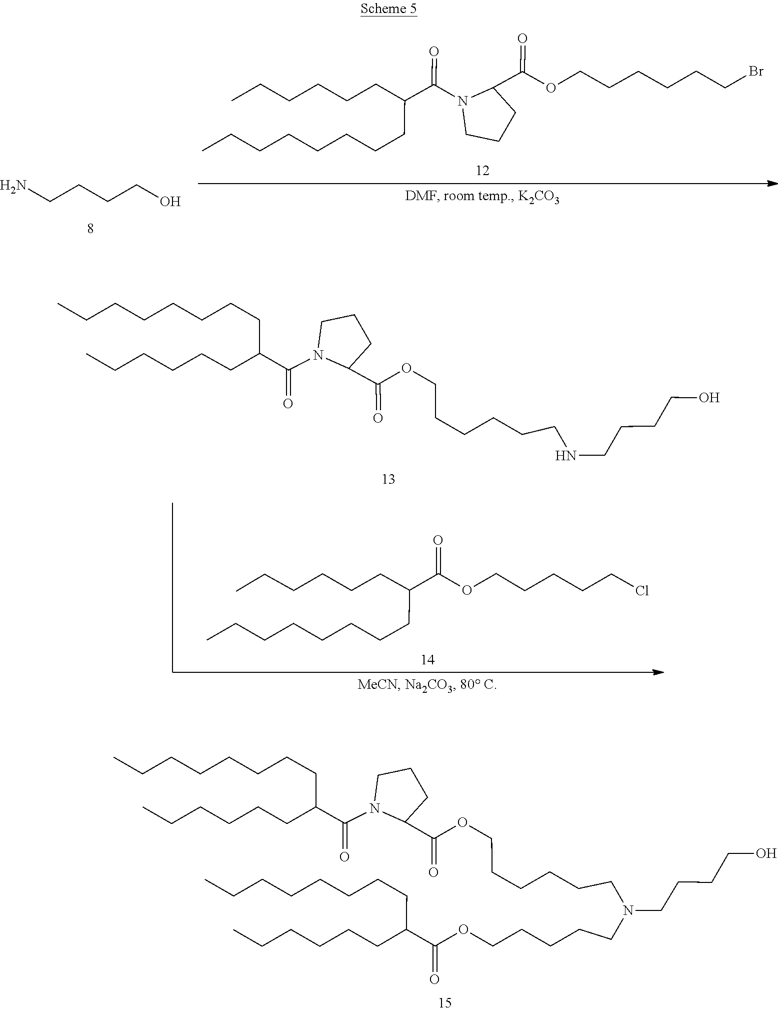 [SMILES: CCCCCCCCC(CCCCCC)C(=O)N1CCCC1C(=O)OCCCCCCBr ; NCCCCO ; CCCCCCCCC(CCCCCC)C(=O)N1CCCC1C(=O)OCCCCCCNCCCCO ; CCCCCCCCC(CCCCCC)C(=O)OCCCCCCl ; CCCCCCCCC(CCCCCC)C(=O)OCCCCCN(CCCCO)CCCCCCOC(=O)C1CCCN1C(=O)C(CCCCCC)CCCCCCCC]

The ionizable amino lipids produced by the method of the disclosure can be in the form of a pharmaceutical salt, such as but not limited to an acid addition salt. Such salts are known to those of skill in the art and include any suitable inorganic acid used in pharmaceutical formulations. Non-limiting examples include acids such as hydrochloric acid, hydrobromic acid, sulfuric acid, phosphoric acid, and the like.

### EXAMPLES

### Example 1: Preparation of ALC-0305 Using the Inventive Method

Experimental section. Unless otherwise specified, all reagents and solvents were commercial products and were used without further purification, except CH₂Cl₂ (freshly distilled from CaH₂ under Ar) and CH₃CN (freshy obtained from a solvent purification system). All reactions were performed under an argon atmosphere. Reaction mixture from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck 60 F254 plates) and column chromatography was performed on 230-400 mesh silica gel. Visualization of the developed chromatogram was performed by staining with I₂ or potassium permanganate solution. ¹H and ¹³C nuclear magnetic resonance (NMR) spectra were recorded at room temperature in CDCl₃ solutions. ¹H NMR spectra (300 MHz for ¹H and 75 MHz for ¹³C) were obtained at room temperature from CDCl₃ solutions. Chemical shifts are reported in parts per million (ppm) on the 8 scale. Multiplicities are reported as “s” (singlet), “d” (doublet), “t” (triplet), “q” (quartet), “m” (multiplet), and further qualified as “app” (apparent) and “br” (broad). Proton NMR spectra were referenced to residual CHCl₃ (7.26 ppm) and ¹³C NMR spectra were referenced to the central line of the CDCl₃ triplet (77.00 ppm). Mass spectra (m/z) were obtained in the electrospray (ESI) mode.

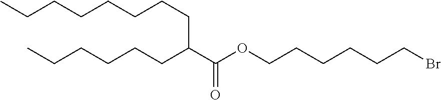 [SMILES: CCCCCCCCC(CCCCCC)C(=O)OCCCCCCBr]

6-Bromohexyl 2-hexyldecanoate, 10. Two drops of 98% sulfuric acid were added to solution of 2-hexvldecanoic acid (15.6 g. 60.8 mmol, 1.1 equiv) and 6-bromohexanol (10 g, 55.2 mmol, 1 equiv) in cyclohexane (30 mL), and the mixture was brought to reflux with continuous removal of water (Dean-Stark trap). After 24 h, some starting alcohol (ca. 10% by 1H NMR) was still present. One drop of 98% H₂SO₄ was added and the mixture was refluxed for another 24 h, whereupon the reaction was complete (¹H NMR). The reaction mixture was cooled to room temperature, diluted with hexanes (20 mL), washed with 5% aqueous Na₂CO₃ solution (2×10 mL), dried (Na₂SO₄) and evaporated. The crude product thus obtained (22 g, 97%) was used directly in the next step. ¹H NMR δ 4.10 (t, 2H, J=6.5), 3.40 (t, 2H, J=6.8), 2.31 (m, 1H), 1.87 (m, 2H), 1.8-1.4 (m, 30H), 0.88 (t, 6H, J=6.2 Hx). ¹³C NMR δ 176.8, 63.8, 45.9 33.6, 32.7, 32.5 (2 peaks), 31.9, 31.7, 29.6, 29.5, 29.2 (2 peaks), 28.5, 27.8, 27.5, 27.4, 25.2, 22.6 (2 peaks), 14.1 (2 peaks). LRMS: m/z 419 [M+H]⁺.

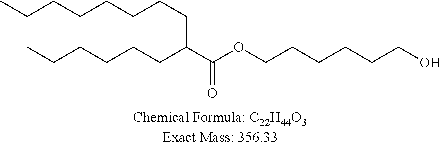 [SMILES: CCCCCCCCC(CCCCCC)C(=O)OCCCCCCO]

6-Hydroxyhexyl 2-hexyldecanoate, 6. A solution of 2-hexyldecanoic acid (10 g, 39.0 mmol, 1 equiv) and 1,6-hexanediol (7.0 g, 58.5 mmol, 1.5 equiv) in toluene (70 mL) was refluxed with continuous removal of water (Dean-Stark trap) for 12 h, whereupon almost all the starting 2-hexyldecanoic acid disappeared. The mixture was cooled to room temperature and washed with deionized water to remove excess 1,6-hexanediol, which was subsequently recovered upon evaporation of the water and recycled. The toluene solution was further washed with 1 N aq. NaOH solution (3×20 mL), then it was percolated through a plug of anhydrous Na₂SO₄ and evaporated. The oily residue was purified by column chromatography on silica gel (5→15% AcOEt in hexanes) to afford 11 g (80%) of pure product. ¹H NMR δ 4.07 (t, 2H, J=6.7 Hz), 3.65 (t, 2H, J=6.3 Hz), 2.30 (m, 1H), 1.70-1.50 (m, 6H), 1.45-36 (br m, 7H), 1.32-1.19 (m, 20H), 0.87 (br t, 6H, J=6.2 Hz). ¹³C NMR δ 176.7, 63.9, 62.8, 45.8, 32.6, 32.5 (2 peaks), 31.8, 31.7, 29.5, 29.4, 29.2 (2 peaks), 28.7, 27.5 (2 peaks), 25.8, 25.4, 22.7 (2 peaks), 14.1 (2 peaks). LRMS: m/z 379 [M+Na]⁺.

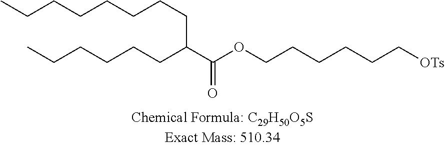 [SMILES: CCCCCCCCC(CCCCCC)C(=O)OCCCCCCOS(=O)(=O)c1ccc(C)cc1]

6-(Tosyloxy)hexyl 2-hexyldecanoate. Solid para-toluenesulfonyl chloride (402 mg, 2.1 mmol, 1.5 equiv) was added to a solution of 6-hydroxyhexyl 2-hexyldecanoate (500 mg, 1.4 mmol, 1 equiv) and triethylamine (285 mg, 392 uL, 2.8 mmol, 2 equiv) in CH₂Cl₂ (1.8 mL) and the resulting mixture was stirred at room temperature overnight. A white precipitate appeared. The solution was sequentially washed with aqueous saturated NaHCO₃ solution, water, and brine, dried (Na₂SO₄), and evaporated to give 570 mg of crude tosylate (80%), which was used without purification. ¹H NMR δ 7.78 (app d, 2H J=8.2 Hz), 7.34 (app d, 2H, J=8.2 Hz), 4.01 (4H, 2 overlapping t, J=6.1 Hz), 2.45 (s, 3H), 2.29 (m, 1H), 1.69-1.51 (m, 8H), 1.47-1.13 (m, 24H) 0.86 (br t, 6H, J=6.3 Hz). ¹³C NMR δ 176.6, 145.0, 133.0, 130.0, 128.2, 70.3, 63.7, 45.8, 32.5, 31.8, 31.7, 29.5 (2 peaks), 29.2 (2 peaks), 28.7, 28.5, 27.4 (2 peaks), 25.4, 25.0, 22.6 (2 peaks), 21.6, 14.1 (2 peaks).

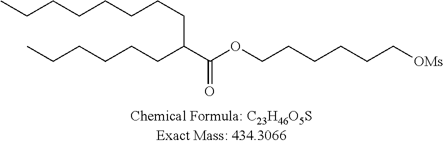 [SMILES: CCCCCCCCC(CCCCCC)C(=O)OCCCCCCOS(C)(=O)=O]

6-(Mesyloxy)hexyl 2-hexyldecanoate, 11. Methanesulfonyl chloride (2.0 g, 1.3 mL, 17 mmol, 1.2 equiv) was added to a solution of 6-hydroxyhexyl 2-hexyldecanoate (5 g, 14 mmol, 1 equiv) and triethylamine (2.2 g, 21 mmol, 2.9 mL, 1.5 equiv) in CH₂Cl₂ (15 mL) and the resulting mixture was stirred at room temperature overnight. A white precipitate appeared. The solution was evaporated and the residue was taken up with hexane. The resulting solution was sequentially washed with aqueous saturated NaHCO₃ solution (3×10 mL), water (2×10 mL), and brine (2×10 mL), dried (Na₂SO₄), filtered through Celite and evaporated to afford 5.3 g (87%) of crude mesylate, which was used without further purification. ¹H NMR δ 4.18 (br t, 2H, J=6.0 Hz), 4.03 (br t, 2H, J=6.0Hz), 2.96 (s, 3H), 2.27 (m, 1H), 1.78-1.2 (m, 32H), 0.83 (br t, 6H, J=6.5 Hz). ¹³C NMR δ 176.5, 69.7, 63.6, 45.7, 37.2, 32.4 (2 peaks), 31.7, 31.6, 29.4, 29.3, 21.1 (2 peaks), 28.9, 28.4, 27.3 (2 peaks), 25.4, 25.0, 22.5 (2 peaks), 13.9 (2 peaks).

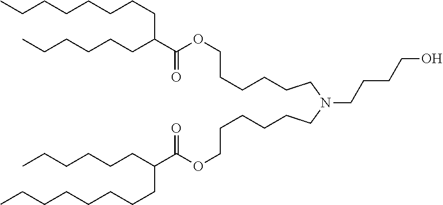 [SMILES: CCCCCCCCC(CCCCCC)C(=O)OCCCCCCN(CCCCO)CCCCCCOC(=O)C(CCCCCC)CCCCCCCC]

((4-Hydroxybutyl)azanediyl)bis(hexane-6,1-diyl) bis(2-hexyldecanoate) (ALC-0315) from bromide 10. A solution of 4-amino-1-butanol (465 mg, 5.2 mmol, 1 equiv) and 6-bromohexyl 2-hexyldecanoate (5.0 g, 11.9 mmol, 2.3 equiv) in dry acetonitrile (25 mL) containing suspended anhydrous Na₂CO₃ (1.2 g, 10.4 mmol, 2.0 equiv) and NaI (560 mg, 2.83 mmol, 0.5 equiv) was heated to 75° C. in a sealed reactor, under argon atmosphere. The reaction was monitored by TLC, MS and ¹H NMR. A lower layer immiscible with MeCN and containing the desired product separated. After 20 hours, the mixture was cooled and diluted with water (10 mL) and hexanes (20 mL). The organic phase was separated and sequentially washed with water (2×10 mL) and brine (10 mL), then dried (Na₂SO₄) and evaporated. The residue was purified by column chromatography (0→3% MeOH in CH₂Cl₂) to afford 2.5 g (63%) of pure product. The same procedure was employed for the reaction of sulfonate derivatives of alcohol 6, such as mesylate 11 or the corresponding tosylate (yield of 3:55-60%). ¹H NMR δ 4.05 (t, 4H, J=6.6 Hz), 3.58 (br m, 2H), 2.53 (br m, 6H), 2.30 (m, 2H), 1.76-1.50 (m, 14H), 1.47-1.12 (m, 54H), 0.87 (br t, 12H, J=6.9 Hz). The OH proton is not visible. ¹³C NMR δ 176.7, 63.9, 62.1, 53.4, 45.8, 32.5 (2 peaks), 31.8, 31.7, 29.5, 29.4, 29.2 (2 peaks), 28.6, 27.4 (2 peaks), 27.1, 25.8, 22.6 (2 peaks), 14.0 (2 peaks). Overlapping resonances reduce the number of unique signals from 26 to 22). LRMS: 766 [M+H]⁺.

### Example 2: Preparation of Ionizable Lipid 15 Using the Inventive Method

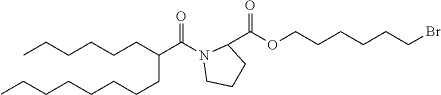 [SMILES: CCCCCCCCC(CCCCCC)C(=O)N1CCCC1C(=O)OCCCCCCBr]

6-bromohexyl-N-(2-hexyldecanoyl)prolinate (12). A solution of N-(2-mmol), 6-bromo-1-hexanol (543 mg, 3.00 mmol), EDCI-HCl (665 mg, 3.47 mmol) and DMAP (424 mg, 3.47 mmol) in DCM (10.0 mL) was stirred under inert atmosphere for 18 hours then concentrated. The residue was purified by silica chromatography (0-20% EtOAc in Hexanes) to yield 12 (995 mg, 78%) as an oil. ¹H NMR (400 MHZ, CDCl₃, rotamers) δ 4.51-4.45 (m, 1H), 4.21-4.03 (m, 2H), 3.71-3.63 (m, 1H), 3.61-3.51 (m, 1H), 3.43-3.39 (m, 2H), 2.49-2.43 (m, 1H), 2.23-1.75 (m, 6H), 1.68-1.59 (m, 4H), 1.49-1.34 (m, 6H), 1.26-1.24 (br m, 20H), 0.89-0.85 (m, 6H). LRMS: 516 & 518 [M+H]⁺.

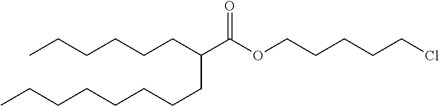 [SMILES: CCCCCCCCC(CCCCCC)C(=O)OCCCCCCl]

5-chloropentyl 2-hexyldecanoate (14). Obtained from 2-hexyldecanoic acid and 5-chloro-1-pentanol by the method described above for 10. ¹NMR (400 MHZ, CDCl₃) δ 4.12-4.08 (t, 2H), 5.67-3.543 (t, 2H), 2.36-2.29 (m, 1H), 1.79-1.86 (m, 2H), 1.71-1.42 (m, 8H), 1.27 (br s, 22H), 0.91-0.87 (m, 6H).

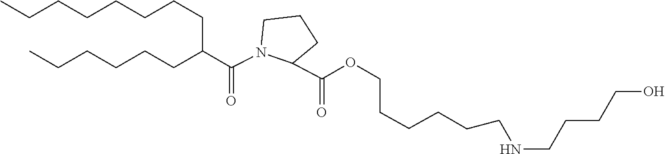 [SMILES: CCCCCCCCC(CCCCCC)C(=O)N1CCCC1C(=O)OCCCCCCNCCCCO]

6-((4-hydroxybutyl)amino)hexyl (2-hexyldecanoyl)prolinate (15). A solution of bromide 12 (229 mg, 0.468 mmol), 4-amino-1-butanol (44 mg, 0.491 mmol) and K₂CO₃ (68 mg, 0.491 mmol) in DMF (5 mL) was stirred at room temperature for 18 hours under nitrogen atmosphere. The mixture was then diluted with water (10 mL) and extracted with Et₂O (3×10 mL). The combined extracts were washed with brine (10 mL), dried (Na₂SO₄) and concentrated. The residue was purified by silica chromatography (0-45% MeOH in DCM) to yield the secondary amine 13 (136 mg, 58%). ¹H NMR (400 MHZ, CDCl₃, rotamers) δ 4.13-4.10 (m, 4H), 3.61 (br t, 1H), 3.12 & 3.01 (s, 3H, rotamers 3:1), 2.65-2.72 (m, 4H), 1.68-1.61 (m, 8H), 1.45-1.26 (m, 30H), 0.89-0.86 (m, 6H).

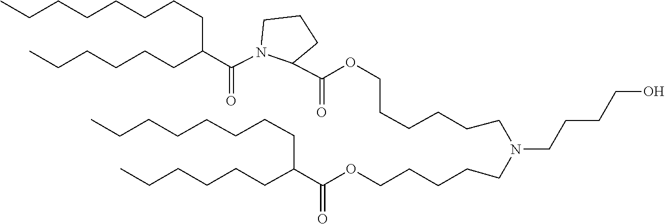 [SMILES: CCCCCCCCC(CCCCCC)C(=O)OCCCCCN(CCCCO)CCCCCCOC(=O)C1CCCN1C(=O)C(CCCCCC)CCCCCCCC]

6-((5-((2-hexyldecanoyl)oxy)pentyl)(4-hydroxybutyl)amino)hexyl (2-hexyl-decanoyl) prolinate (15). A mixture of 13 (126 mg, 0.252 mmol), chloride 14 (116 mg, 0.278 mmol) and K₂CO₃ (42 mg, 0.302 mmol) in MeCN (2.5 mL) was stirred at 80° C. in a sealed reaction vessel for 18 hours. The mixture was cooled, diluted with water (5 mL) and extracted with DCM (3×5 mL). The combined extracts were dried (Na₂SO₄) and concentrated. The residue was purified by silica chromatography (0-5% MeOH in DCM) to yield lipid 15 (135 mg, 64%) as an oil. ¹H NMR (400 MHZ, CDCl₃, rotamers) δ 4.51-4.45 (m, 1H), 4.21-4.03 (m, 4H), 3.71-3.63 (m, 1H), 3.61-3.51 (m, 1H), 3.48 (br m, 2H), 2.85-2.30 (m, 8H), 2.23-1.78 (m, 14H), 1.75-1.20 (m, 56H), 0.89-0.85 (m, 12H).

1. A method for producing an ionizable amino lipid, or a pharmaceutically acceptable salt thereof, the method comprising a nucleophilic displacement of a leaving group L in a compound of Formula II, 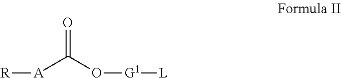
wherein R is a linear or branched C₈ to C₃₀ alkyl group, optionally comprising heteroatoms selected from N, O and/or S, optionally comprising 1-3 C═C double bonds of Z or E geometry, optionally comprising cyclic structures, and/or optionally comprising amide and/or ester linkages,
A is absent or present, and, when present, is a moiety derived from an aminoacid, optionally selected from proline, sarcosine, alanine, beta-alanine and optionally representable as Formula III: 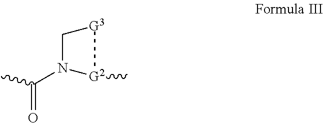
wherein G² and G³ are bonded to each other or not bonded, and
if G² and G³ are bonded to each other, then
G² is CH,
G² is bonded to the carbonyl (C═O) group in Formula II,
G³ is (CH₂)ₙ wherein n is 1 to 4, and
the C═O group in Formula III is bonded to the R group in Formula II, and
if G² and G³ are not bonded, then
G² is (CH₂)ₚ wherein p is 1 to 3,
G² is bonded to the carbonyl (C═O) group in Formula II,
G³ is (CH₂)_{q}-H wherein q is 0 to 3, and
the C═O group in Formula III is bonded to the R group in Formula II,
G¹ is (CH₂)ₙ, wherein n is 2 to 10,
wherein the leaving group L is a halogen atom selected from chlorine, bromine, or iodine or a sulfonate selected from a tosylate or a mesylate.
wherein the nucleophilic displacement comprises reacting a compound of Formula II with an amino alcohol in a single alkylation reaction, resulting in an N-double alkylation of the amino alcohol by the compound of Formula II to form the ionizable amino lipid; or
wherein the nucleophilic displacement comprises reacting two different compounds of Formula II in sequential displacement reactions, a first reaction comprising reacting the amino alcohol with a first compound of Formula II to produce a single N-alkylated compound and alkylating the single N-alkylated compound so produced in a second reaction with a second compound of Formula II to produce the ionizable lipid.

2. The method of claim 1, wherein n of (CH₂)ₙ in G¹ is 2 to 8.

3. The method of claim 1, wherein R is a branched chain that comprises two hydrophobic carbon chains, R¹ and R², each of the chains being independently linear, unsubstituted C₂ to C₁₂ alkyl with 0 double bonds and wherein a carbon branch point is at an alpha, beta or gamma position relative to the carbonyl group of Formula II.

4. The method of claim 1, wherein the nucleophilic displacement comprises reacting a compound of Formula II with an amino alcohol in the single alkylation reaction and wherein Formula II has the structure of Formula I below:

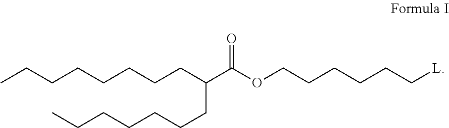

5. The method of claim 1, wherein the nucleophilic displacement comprises reacting the first and second compounds of Formula II in the sequential displacement reactions and wherein the first or second compound of Formula II has a structure of Formula IV below: 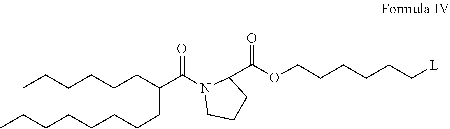
and wherein the first or second compound of Formula II has a structure as in Formula I

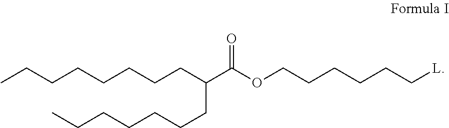

6. The method of claim 1, wherein the amino alcohol is 4-amino-1-butanol.

7. The method of claim 1, wherein the nucleophilic displacement is carried out in a polar solvent with a base and optionally with a source of iodide ion.

8. The method of claim 7, wherein the base is a metal carbonate.

9. The method of claim 8, wherein the optional source of iodide ion is lithium iodide, sodium iodide, potassium iodide, or a tetraalkylammonium iodide.

10. The method of claim 1, further comprising producing the compound of Formula II via an esterification reaction between a carboxylic acid having the structure of Formula V, wherein R¹ and R² are independently substituted, unsubstituted, branched or unbranched C₁-C₂₀ alkyl having 0 to 3 double bonds, and an alcohol with a terminal leaving group L as defined by the structure of Formula VI, wherein G¹ is (CH₂)ₙ, and wherein n is 2 to 10 and L is the leaving group

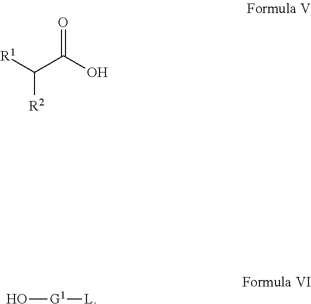

11. The method of claim 1, further comprising producing the compound of Formula II via an esterification reaction between a carboxylic acid having a structure of Formula V, wherein R¹ and R² are independently substituted, unsubstituted, branched or unbranched C₁-C₂₀ alkyl having 0 to 3 double bonds, and a hydroxyl group of a diol having the structure of Formula VII, wherein G¹ is (CH₂)ₙ, and wherein n is 2 to 10; and wherein 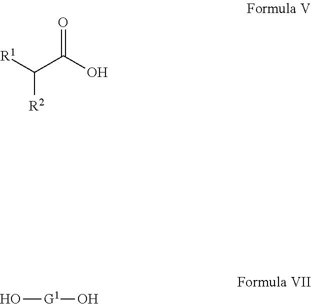
the esterification reaction produces a compound having a structure of Formula VIII: 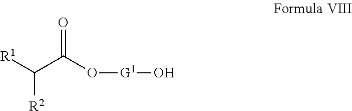
and converting the OH of the compound of Formula VIII into the leaving group L to produce a compound having a structure of Formula II.

12. The method of claim 10, wherein the esterification reaction is a Fischer esterification that is carried out in an inert solvent capable of forming an azeotrope with water.

13. The method of claim 10, wherein the inert solvent is toluene, cyclohexane, 1,2-dichloroethane.

14. The method of claim 10, wherein water produced during the esterification is removed such that the carboxylic acid serves as an acid catalyst for the esterification reaction.

15. The method of claim 10, wherein an acid catalyst is used in the esterification, the acid catalyst selected from sulfuric acid, phosphoric acid, para-toluenesulfonic acid, sodium hydrogen sulfate, or a sulfonated polystyrene resin.

16. The method of claim 1, wherein the nucleophilic displacement comprises the single alkylation and wherein the ionizable lipid produced is ALC-0315.

17. An intermediate for preparing an amino ionizable lipid, the intermediate having a structure of Formula I: 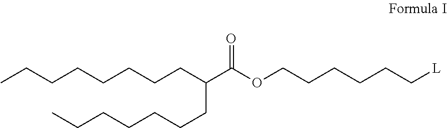
wherein L is a halogen atom selected from chlorine, bromine, or iodine or a sulfonate selected from a tosylate or a mesylate.

18. An intermediate for preparing an amino ionizable lipid, the intermediate having a structure of Formula IX or Formula X, 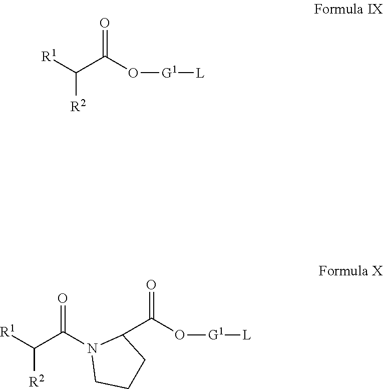
wherein L is a halogen atom selected from chlorine, bromine, or iodine or a sulfonate selected from a tosylate or a mesylate, and wherein G¹ is (CH₂)ₙ, wherein n is 2 to 10.

19. (canceled)

20. (canceled)

21. A method for preparing the intermediate of claim 18 comprising:
(a) an esterification between a hydroxyl group and a carboxylate group of the compounds of Formula V and Formula VI to produce the intermediate 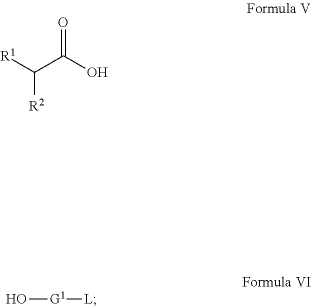
or
(b) an esterification between a hydroxyl group and a carboxylate group of the compounds of Formula V and Formula VII to produce a compound of Formula VIII and further reacting the compound of Formula VIII to convert the terminal hydroxyl group into a leaving group to produce the intermediate

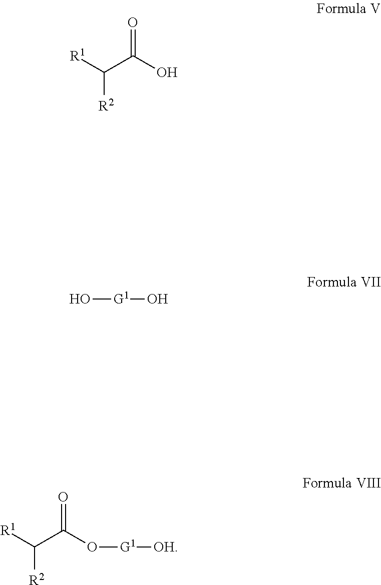

22. The method of claim 21, wherein the esterification reaction is a Fischer esterification that is carried out in an inert solvent capable of forming an azeotrope with water.

23. (canceled)

24. (canceled)

25. (canceled)
